(19) United States
(12) Patent Application Publication
(10) Pub. No.: US 2007/0082934 A1
(43) Pub. Date: Apr. 12, 2007

(54) Substituted 4-aryloxy and 4-arylsulfanyl-phenyl-2-aminothiazoles as inhibitors of cell proliferation

(76) Inventors: Michael J. Gorczynski, Winston-Salem, NC (US); John H. Bushweller, Charlottesville, VA (US); Milton L. Brown, Charlottesville, VA (US)

Correspondence Address: UNIVERSITY OF VIRGINIA PATENT FOUNDATION, 250 WEST MAIN STREET, SUITE 300, CHARLOTTESVILLE, VA, 22902, US

(21) Appl. No.: 10/576,830

(22) PCT Filed: Oct. 27, 2004
(86) PCT No.: PCT/US04/35586

§ 371 Date: Apr. 24, 2006

Related U.S. Application Data
(60) Provisional application No. 60/514,678, filed on Oct. 27, 2003.

(51) Int. Cl. A61K 31/426; C07D 277/18

(52) U.S. Cl. 514/370; 548/190

(57) ABSTRACT

The invention discloses compounds which are substituted 4-aryloxy and 4-arylsulfanyl-phenyl-2-aminothiazoles with anti-cancer activity. The invention futher discloses methods of preparing compounds of the invention. The invention also discloses methods of inhibiting cell proliferation and tumor growth in a subject by administering compounds of the invention to the subject.

### CROSS REFERENCE TO RELATED APPLICATIONS

The present application claims priority under 35 U.S.C. § 119(e) to U.S. Provisional Application No. 60/514,678 filed on Oct. 27, 2003.

### FIELD OF THE INVENTION

The invention relates to methods for preparing substituted 4-aryloxy and 4-arylsulfanyl-phenyl-2-aminothiazoles and for the use of substituted 4-aryloxy and 4-arylsulfanyl-phenyl-2-aminothiazoles as inhibitors of cell proliferation.

### BACKGROUND

Breast cancer is the most common cancer and second most common cause of cancer death among women. The National Cancer Institute (NCI) estimates 211,000 new breast cancer cases and 40,000 deaths for 2003. There are two types of breast cancer: in situ and invasive. In situ breast cancer is divided into two subtypes: ductal cystic (DCIS) and lobular cystic (LCIS). Infiltrating ductal carcinoma is the most common type of invasive breast cancer accounting for approximately 70% of all breast cancer diagnoses. Another type of invasive breast cancer is infiltrating lobular carcinoma. This form accounts for 5-10% of invasive breast cancers. In addition, there are a few less common histologic subtypes of invasive cancer including medullary, mucinous, and tubular.

Current treatments for breast cancer are based on the patient's type of breast cancer. Local incision plus radiation or a simple mastectomy are possible treatment options for patients with DCIS. There is a 20-30% risk of developing invasive breast cancer for women with LCIS and as a result, it is managed with careful bilateral breast observation.

Invasive cancers are treated surgically by either a modified radical mastectomy with axiliary lymph node dissection or a lumpectomy with axiliary lymph node dissection followed by local radiation. Surgical treatment options are standard treatment therapies for DCIS and invasive cancers followed by post-surgical (adjuvant) treatment. The aforementioned adjuvant therapies have improved survival rates of women with these types of breast cancer. In addition to surgical options, adjuvant drug therapy can decrease the risk of systemic recurrence by approximately one third.

Adjuvant treatments include cytotoxic chemotherapy (i.e. paclitaxel, doxorubicin), hormonal therapy (i.e. tamoxifen), or a combination of the two: These treatments are not without severe side effects. Paclitaxel inhibits microtubule disassembly and is active against a wide range of human tumors. The major side effects of paclitaxel are neutropenia, neurotoxicity, and cardiotoxicity. Doxorubicin is one of the most active cytotoxic agents against breast cancer, but side effects, such as cardiotoxicity and myelosuppression, have hindered this drug's use in adjuvant therapy. Tamoxifen is the only drug approved by the United States Food and Drug Administration for breast cancer risk-reduction in estrogen-sensitive breast cancer. Women receiving tamoxifen may experience more frequent hot flashes, the development of cataracts, and increased risk for venous thromboembolic events and strokes. Tamoxifen use is also associated with increased endometrial cancer risk in postmenopausal women with a uterus. Because of the side effects caused by the current adjuvant treatments, there is a need for safer, more potent breast cancer agents.

2-aminothiazoles represent a fairly new class of breast cancer drugs with only a few examples in the literature (FIG. 1). Currently, a number of aminothiazolecarbonitriles (Bilodeau et al., U.S. Pat. No. 6,586,424) and thiazolylaminopyridines (Bilodeau and Hartman, U.S. Patent Publication 20030100567; Bilodeau et al., U.S. Patent Publication 20020137755) are being investigated for their use as tyrosine kinase inhibitors. Aminothiazole inhibitors of cyclin-dependent kinase 2 have been shown to have significant antitumor activity in breast cancer models (Kim et al., J. Med. Chem. 2002, 45:18:3905-3927).

Thiophene-2 carboxamidines containing 2-aminothiazoles have been evaluated as serine protease urokinase inhibitors (Wilson et al., Bioorg. Med. Chem. Lett. 2001, 11:7:915-918). In addition to 2-aminothiazoles, there are also few examples in the breast cancer literature of compounds containing diaryl ethers. A number of diaryl ether compounds have been evaluated as anti-cancer agents (Tuse et al., U.S. Pat. No. 6,169,104), however, there are no reports of diaryl ethers with a 2-aminothiazole moiety.

There is a long felt need in the art for the development of substituted 4-aryloxy-and 4-arylsulfanyl-phenyl-2-aminothiazoles with anti-proliferative activity against cancer cells. The present invention satisfies these needs.

### SUMMARY OF THE INVENTION

The present invention, as described in the disclosure provided herein, is based On the discovery that substituted 4-aryloxy-and 4-arylsulfanyl-phenyl-2-aminothiazoles have anti-proliferative activity. The summary, as well as the following detailed description of preferred embodiments of the invention, will be better understood when read in conjunction with the chemical structures and formulas provided herein. For the purpose of illustrating the invention, there are shown by chemical structures and formulas embodiments which are presently preferred. It should be understood, however, that the invention is not limited to the precise arrangements and instrumentalities provided herein.

In one aspect, compounds of the present invention are analogs, derivatives, or modifications of structure I:

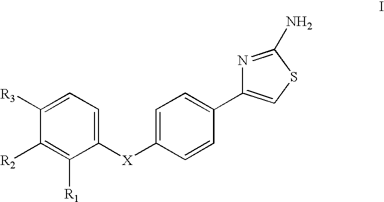

In another aspect, the invention is directed to substituted 4-aryloxy-and 4-arylsulfanyl-phenyl-2-aminothiazoles compounds according to structure I, and to analogs, modifications and derivatives thereof selected from the group consisting of compounds 16 to 30. Compounds 16 to 30 of the present invention are:

4-(4′-Phenoxyphenyl)-thiazol-2-yl ammonium iodide (16); 4-[4′-(4-Chlorophenoxy)-phenyl]-thiazol-2-yl ammonium iodide (17); 4-[4-(3′-Chlorophenoxy)-phenyl]-thiazol-2-yl ammonium iodide (18); 4-[4-(2′-Chlorophenoxy)-phenyl]-thiazol-2-yl ammonium iodide (19); 4-[4′-(3,4-Dichlorophenoxy)-phenyl]-thiazol-2-yl ammonium iodide (20); 4-[4′-(4; Methoxyphenoxy)-phenyl]-thiazol-2-yl ammonium iodide (21); 4-[4′-Cv-Toluoxy)phenyl]-thiazol-2-yl ammonium iodide (22); 4-[4′-(Biphenyl-4-yloxy)-phenyl]-thiazol-2-yl ammonium iodide (23); 4-[4′-(4-Phenoxy-phenoxy)-phenyl]-thiazol-2-yl ammonium iodide (24); 4-[4-(3′-Ethoxycarbonyl-phenoxy)-phenyl]-thiazol-2-yl ammonium iodide (25); 4-(4′-Phenylsulfanyl-phenyl)-thiazol-2-yl ammonium iodide (26); 4-[4-(4′-Chloro-phenylsulfanyl)-phenyl]-thiazol-2-yl ammonium iodide (27); 4-[4-(3′,4′-Dichloro-phenylsulfanyl)-phenyl]-thiazol-2-yl ammonium iodide (28); 4-[4-(4′-Methoxy-phenylsulfanyl)-phenyl]-thiazol-2-yl ammonium iodide (29); and 4-(4′p -Tolylsulfanyl-phenyl)-thiazol-2-yl ammonium iodide (30).

In one aspect, compounds of the invention can be used to inhibit cell proliferation. In another aspect, the cell is a cancer cell. In a further aspect, the cancer cell is selected from the group consisting of breast cancer, leukemia, non-small cell lung cancer, colon cancer, cancer of the central nervous system, melanoma, ovarian cancer, renal cancer, and prostate cancer. In one aspect of the invention, the breast cancer cell is selected from the group consisting of estrogen receptor (ER) positive, ER negative, and adriamycin-resistant breast cancer cells.

In one aspect of the invention, compounds of the invention are useful as anti-cancer agents. In another aspect of the invention, the compounds are useful anti-cancer agents against cancers selected from the group consisting of breast cancer, leukemia, non-small cell lung cancer, colon cancer, cancer of the central nervous system, melanoma, ovarian cancer, renal cancer, and prostate cancer. In one aspect of the invention, the breast cancer is selected from the group consisting of estrogenreceptor (ER) positive, ER negative, and adriamycin-resistant breast cancer.

In one aspect, compounds of the invention are prepared according to Scheme I, as provided herein (see detailed description).

### BRIEF DESCRIPTION OF THE DRAWINGS

The foregoing summary, as well as the following detailed description of preferred embodiments of the invention, will be better understood when read in conjunction with the appended drawings. For the purpose of illustrating the invention, there are shown in the drawings embodiments which are presently preferred. It should be understood, however, that the invention is not limited to the precise arrangements and iumentalities shown. In the drawings:

FIG. 1 illustrates several examples of current 2-aminothiazoles being investigated for breast cancer activity.

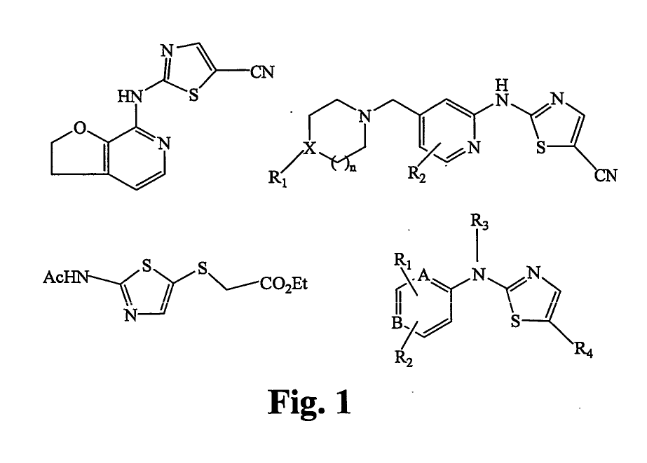

FIG. 2 is a schematic of the general structure of 4-aryloxy- and 4-arylsulfanyl-phenyl-2-aminothiazole salts.

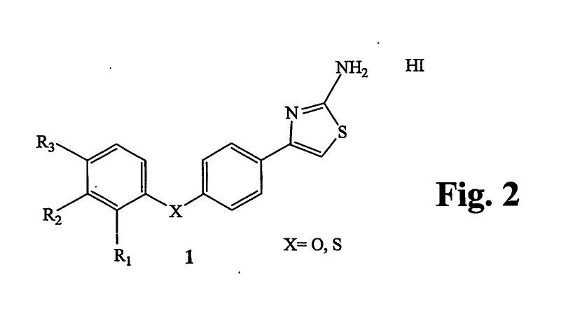

FIG. 3 is a graphic representation of a GI₅₀ comparison for compound 17 (4-[4′-(4-Chlorophenoxy)-phenyl]-thiazol-2-yl ammonium iodide) for all NCI cell lines. The ordinate represents growth inhibitor activity and the abscissa indicates the type of tumor as well as each cell line treated with compound 17. L=leukemia; NSCLC Non-small cell lung cancer; C=Colon; CNS=central nervous system; M=Melanoma; O=Ovarian; R=Renal; P=Prostate; B=Breast.

FIG. 4 illustrates Scheme 1, a scheme for synthesis of substituted acetophenones and thiazoles. Reagents and conditions: a) phenol or thiol, K₂CO₃, DMAC, reflux, 8-10 h, 38-93%; b) thiourea, I₂, EtOH, 100° C., 3 h, 40-98%

### DETAILED DESCRIPTION OF THE INVENTION

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described herein.

As used herein, each of the following terms and acronyms has the meaning associated with it in this section.

The articles “a” and “an” are used herein to refer to one or to more than one (i.e., to at least one) of the grammatical object of the article. By way of example, “an element” means one element or more than one element.

“Anti-cancer activity,” as used herein, refers to inhibiting cancer cell growth in vitro or in vivo and to inhibiting tumor growth in vivo. An “anti-cancer agent”, as used herein, refers to an agent with anti-cancer activity.

A “compound,” as used herein, refers to any type of substance or agent that is commonly considered a drug, or a candidate for use as a drug, as well as combinations and mixtures of the above, or modified versions, analogs, or derivatives of the compound.

A “derivative compound,” as used herein, refers to a compound derived from substituted 4-aryloxy-and 4-arylsulfanyl-phenyl-2-aminothiazoles, said derivative compound having the basic propofal structural formula (i.e., structural formula I) which has been prepared according to the description provided herein. A “derivative compound” as used herein includes compounds which incorporate the basic structural formula of substituted 4-aryloxy-and 4-arylsulfanyl-phenyl-2-aminothiazoles (see structure I and scheme 1) as described herein.

An “effective amount” or “therapeutically effective amount” of a compound is that amount of compound which is sufficient to provide a beneficial or selected effect to the subject to which the compound is administered. For example, an effective amount of substituted 4-aryloxy-and 4-arylsulfanyl-phenyl-2-aminothiazoles, or analogs, derivatives, or modifications thereof, is an amount of the compound sufficient to inhibit the growth of a tumor or to reduce the rate of growth of a tumor in a patient receiving the dose amount.

ER means estrogen receptor. ER positive indicates an estrogen receptor positive tumor or cell, while ER negative indicates an estrogen receptor negative tumor or cell.

As used herein, a “functional” compound or molecule is a compound or molecule in a form in which it exhibits a property by which it is characterized. A functional substituted 4-aryloxy-and 4-arylsulfanyl-phenyl-2-aminothiazoles derivative is one which has the biological properties of structure I, namely, “anticancer activity.”

“Inhibiting cell proliferation” and “inhibiting tumor growth” as described herein, refers to any method or technique which inhibits cell proliferation or tumor growth. Inhibition can be direct or indirect.

As used herein, an “instructional material” includes a publication, a recording, a diagram, or any other medium of expression which can be used to communicate the usefulness of the composition of the invention for its designated use. The instructional material of the kit of the invention may, for example, be affixed to a container which contains the composition or be shipped together with a container which contains the composition. Alternatively, the instructional material may be shipped separately from the container with the intention that the instructional material and the composition be used cooperatively by the recipient.

“Modified” compound, as used herein, refers to a modification or derivation of a compound, which may be a chemical modification, such as in chemically altering a compound in order to increase or change its functional ability or activity.

The term, “parenteral” means not through the alimentary canal, but by some other route such as subcutaneous, intramuscular, intraspinal, or intravenous.

As used herein, the term “pharmaceutically-acceptable carrier” means a chemical composition with which an appropriate compound or derivative can be combined and which, following the combination, can be used to administer the appropriate compound to a subject. The term “pharmaceutically acceptable carrier” encompasses any of the standard pharmaceutical carriers, such as a phosphate buffered saline solution, water and emulsions such as an oil/water or water/oil emulsion, and various types of wetting agents.

As used herein, the term “physiologically acceptable” ester or salt means an ester or salt form of the active ingredient which is compatible with any other ingredients of the pharmaceutical composition, which is not deleterious to the subject to which the composition is to be administered.

As used herein, the term “purified” and like terms relate to the isolation of a molecule or compound in a form that is substantially free of contaminants normally associated with the molecule or compound in a native or natural environment.

As used herein, the term “treating” includes prophylaxis of the specific disorder or condition, or alleviation of the symptoms associated with a specific disorder or condition and/or preventing or eliminating said symptoms.

A “subject” which is administered a compound of the invention is a mammal, including a human. Non-human animals subject to administration of a compound of the invention include, but are not limited to, primates, cats, dogs, horses, cows, goats, and sheep.

The term “substantially pure” describes a compound, e.g., substituted 4-aryloxy-and 4-arylsulfanyl-phenyl-2-aminothiazoles derivatives, or analogs or modifications thereof, which has been separated from components which naturally accompany it during synthesis. Typically, a compound is substantially pure when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method.

“Synthesis of substituted 4-aryloxy-and 4-arylsulfanyl-phenyl-2-aminothiazoles,” as used herein, refers to the formation or production of a derivative of the basic structural formula I, or modifications or analogs thereof.

The term to “teat,” as used herein, means reducing the frequency with which symptoms are experienced by a patient or subject or administering an agent or compound to reduce the frequency with which symptoms are experienced.

As used herein, “treating a disease or disorder” means reducing the frequency with which a symptom of the disease or disorder is experienced by a patient. Disease and disorder are used interchangeably herein.

Chemical Groups

As used herein the term “aryl” refers to a mono- or bicyclic carbocyclic ring system having one or two aromatic rings including, but not limited to, phenyl, benzyl, naphthyl, tetrahydronaphthyl, indanyl, indenyl, and the like. “Optionally substituted aryl” includes aryl compounds having from zero to four substituents, and “substituted aryl” includes aryl compounds having one to three substituents. The term (C₅-C₈ alkyl)aryl refers to any aryl group which is attached to the parent moiety via the alkyl group.

The term “bicyclic” represents either an unsaturated or saturated stable 7- to 12-membered bridged of fused bicyclic carbon ring. The bicyclic ring may be attached at any carbon atom which affords a stable structure. The term includes, but is not limited to, naphthyl, dicyclohexyl, dicyclohexenyl, and the like.

The term “C₁-Cₙ alkyl” wherein n is an integer, as used herein, represents a branched or linear alkyl group having from one to the specified number of carbon atoms. Typically, C₁-C₆ alkyl groups include, but are not limited to, methyl, ethyl, n-propyl, iso-propyl, butyl, iso-butyl, sec-butyl, tert-butyl, pentyl, hexyl, and the like.

The term “C₂-Cₙ alkenyl” wherein n is an integer, as used herein, represents an olefinically unsaturated branched or linear group having from 2 to the specified number of carbon atoms and at least one double bond. Examples of such groups include, but are not limited to, 1-propenyl, 2-propenyl, 1,3-butadienyl, 1-butenyl, hexenyl, pentenyl, and the like.

The term “C₂-Cₙ allynyl” wherein n is an integer refers to an unsaturated branched or linear group having from 2 to the specified number of carbon atoms and at least one triple bond. Examples of such groups include, but are not limited to, 1-propynyl, 2-propynyl, 1-butynyl, 2-butynyl, 1-pentynyl, and the like.

The term “C₃-Cₙ cycloalkyl” wherein n is an integer refers to cyclic non-aryl group, for example C₃-C₈ cycloalkyl, represents cyclopropyl, cyclobutyl, cyclopentyl, cyclohexyl, cycloheptyl, and cyclooctyl.

As used herein, the term “halo” means Cl, Br, F, and I. Especially preferred halogens include Cl, Br, and F. The term “haloalkyl” as used herein refers to a C₁-Cₙ alkyl radical bearing at least one halogen substituent, for example, chloromethyl, fluoroethyl or trifluoromethyl and the like.

As used herein the term “heteroaryl” refers to a mono- or bicyclic carbocyclic ring system having one or two aromatic rings containing from one to three heteroatoms and includes, but is not limited to, furyl, thienyl, pyridyl and the like.

The term “heterocyclic group” refers to a mono- or bicyclic carbocyclic ring system containing from one to three heteroatoms wherein the heteroatoms are selected from the group consisting of oxygen, sulir, and nitrogen.

The term “lower alkyl” as used herein refers to branched or straight chain alkyl groups comprising one to eight carbon atoms, including methyl, ethyl, propyl, isopropyl, n-butyl, t-butyl, neopentyl, and the like.

As used herein, the term “optionally substituted” refers to from zero to four substituents, wherein the substituents are each independently selected. Each of the independently selected substituents may be the same or different than other substituents.

Compounds of the present invention that have one or more asymmetric carbon atoms may exist as the optically pure enantiomers, or optically pure diastereomers, as well as mixtures of enantiomers, mixtures of diastereomers, and racemic mixtures of such stereoisomers. The present invention includes within its scope all such isomers and mixtures thereof.

Several substituted 4-aryloxy- and 4-arylsulfanyl-phenyl-2-aminothiazoles have been synthesized herein and evaluated for cytotoxic and growth inhibitory activity against estrogen-positive, estrogen negative, and adriamycin-resistant human breast cancer cell lines. For example, 4-[4′-′(3,4-Dichlorophenoxy)-phenyl]-thiazol-2-yl ammonium iodide (compound 20) demonstrated potent activity against both estrogen-positive and negative breast cancer cell lines with low micromolar (μM) GI₅₀ values. In addition, several 2-aminothiazoles are provided herein that demonstrate selective potency for adriamycin-resistant and estrogen-negative breast cancer cell lines. Without wishing to be bound by any particular theory, the results suggest that these 2-aminothiazoles represent lead compounds for evaluation in animal models of breast cancer and other forms of hormone dependent and independent cancer.

In accordance with the present invention, an anticancer agent is provided wherein the agent has the general structure: 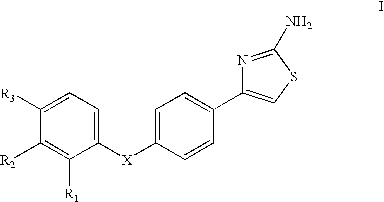
wherein X is selected from the group consisting of O, S, and NH, and R₁, R₂, and R₃ are independently selected from the group consisting of H, halo, (C₁-C₄)alkyl, (C₁-C₄)alkoxy, aryl, —O-aryl and (CO)OR4, wherein R4 is H or (C₁-C₄)alkyl. In one embodiment, X is O or S, R₁ is H, and R₂, and R₃ are independently selected from the group consisting of H, halo, (C₁-C₄)alkyl, (Cr=C₄)alkoxy, phenyl, —O-phenyl and (CO)OR4, wherein R4 is (C₁-C₄)alkyl. In another embodiment the compounds has the general structure of Formula I, wherein X is O or S, R₁ is H, and R₂, and R₃ are independently selected from the group consisting of H, Cl, (C₁-C₂)alkyl, (C₁-C₂)alkoxy, phenyl, —O-phenyl and (CO)OCH₂CH₃ and pharmaceutically acceptable salts thereof.

In one embodiment, a compound of the present invention is selected from the group consisting of compounds 16-30 as described herein, or analogs, derivatives and modifications thereof. Compounds 16-30 are named, respectively:

4-(4′-Phenoxyphenyl)-thiazol-2-yl ammonium iodide (16); 4-[4′-(4-Chlorophenoxy)-phenyl]-thiazol-2-yl ammonium iodide (17); 4-[4-(3′-Chlorophenoxy)-phenyl]-thiazol-2-yl ammonium iodide (18); 4-[4-(2′-Chlorophenoxy)-phenyl]-thiazol-2-yl ammonium iodide (19); 4-[4′-(3,4-Dichlorophenoxy)-phenyl]-thiazol-2-yl ammonium iodide (20); 4-[4′-(4-Methoxyphenoxy)-phenyl]-thiazol-2-yl ammonium iodide (21); 4-[4′-(4-Tolueno)phenyl]-thiazol-2-yl ammonium iodide (22); 4-[4′-(Biphenyl-4-yloxy)-phenyl]-thiazol-2-yl ammonium iodide (23); 4-[4′-(4-P1henox-phenoxy)-phenyl] thiazol-2-yl ammonium iodide (24); 4-[4-(3-′-Ethoxycarbonyl-phenoxy)-phenyl]-thiazol-2-yl ammonium iodide (25); 4-(4′-Phenylsulfanyl-phenyl)-thiazol-2-yl ammonium iodide (26); 4-[4-(4′-Cloro-phenylsulfanyl)-phenyl]-thiazol-2-yl ammonium iodide (27); 4-[4-(3′,4′-Dichloro-phenylsulfanyl)-phenyl]-thiazol-2-yl ammonium iodide (28); 4-[4-(4′-Methoxy-phenylsulfanyl)-phenyl]-thiazol-2-yl ammonium iodide (29); and 4-(4′-p-Tolylsulfanyl-phenyl)-thiazol-2-yl ammonium iodide (30).

In one aspect, the compounds of the invention have anti-proliferative activity and inhibit cancer cell proliferation. Preferably, cell proliferation is inhibited by at least 10% when cancer cells are contacted with a compound of the invention, relative to similar cancer cells not contacted with the compound. More preferably, cell proliferation is inhibited by at least 25% when cancer cells are contacted with a compound of the invention, relative to similar cancer cells not contacted with the compound. Even more preferably, cell proliferation is inhibited by at least 50% when cancer cells are contacted with a compound of the invention, relative to similar cancer cells not contacted with the compound. Most preferably, cell proliferation is inhibited by at least 75% when cancer cells are contacted with a compound of the invention, relative to similar cancer cells not contacted with the compound.

One embodiment of the present invention is directed to pharmaceutical compositions comprising the compounds of the invention and a pharmaceutically acceptable carrier. The pharmaceutically acceptable carrier can be selected from among the group consisting of excipients, disintegrating agents, binders and lubricating agents. The amount of the pharmaceutical agent suitable for administration will be in accordance with standard clinical practice. In addition, the pharmaceutical compositions can be further combined with other known anti-cancer agents and used in conjunction with known anti-cancer therapies.

The anticancer compositions of the present invention can be administered either orally or parenterally. In one embodiment, the composition is administered locally by injection or by an implantable time-release device. Local administration includes intratumoral administration. When administered orally, the compounds can be administered as a liquid solution, powder, tablet, capsule, or lozenge. The compounds can be used in combination with one or more conventional pharmaceutical additives or excipients used in the preparation of tablets, capsules, lozenges and other orally administrable forms. When administered parenterally, and more preferably by intravenous injection, the sodium channel blockers of the present invention can be admixed with saline solutions and/or conventional IV solutions.

In accordance with one embodiment of the present invention, a method is provided for inhibiting the proliferation of cancer cells, and more particularly in one embodiment, breast cancer cells. In one aspect, the cancer cells are selected from the group consisting of breast cancer, leukemia, non-small cell lung cancer, colon cancer, cancer of the central nervous system, melanoma, ovarian cancer, renal cancer, and prostate cancer cells. In one aspect of the invention, the breast cancer cell is selected from the group consisting of estrogen receptor (ER) positive, ER negative, and adriamycin-resistant breast cancer cells. The method comprises contacting the cells with a compound represented by the general structure: 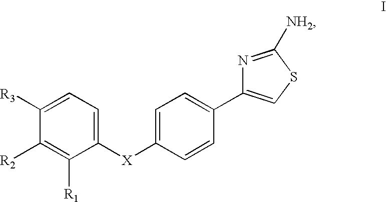
wherein X is selected from the group consisting of O, S, and NH, and R₁, R₂, and R₃ are independently selected from the group consisting of H, halo, (C₁-C₄)alkyl, (C₁-C₄)alkoxy, aryl, —O-aryl and (CO)OR₄, wherein R₄ is H or (C₁-C₄)alkyl. In one embodiment, X is O or S, R₁ is H and R₂, and R₃ are independently selected from the group consisting of H, halo, (C₁-C₄)alkyl, (C₁-C₄)alkoxy, phenyl, —O-phenyl, and (CO)OR4, wherein R4 is (C₁-C₄)alkyl. In another embodiment the compounds of the invention have the general structure of Formula I, wherein X is O or S, R₁ is H, and R₂ and R₃ are independently selected from the group consisting of H, Cl, (C₁-C₂)alkyl, (C₁-C₂)alkoxy, phenyl, —O-phenyl and (CO)OCH₂CH₃, and pharmaceutically acceptable salts thereof.

In accordance with one embodiment of the present invention, a method is provided for treating a mammalian subject, including humans, afflicted by a cancer, such as breast cancer. The method comprises the steps of administering to such a subject an effective amount of a composition comprising a compound represented by the general structure: 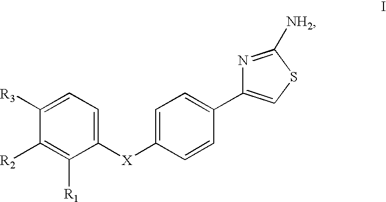
wherein X is selected from the group consisting of O, S, and NH, and R₁, R₂, and R₃ are independently selected from the group consisting of H, halo, (C₁-C₄)alkyl, (C₁-C₄)alkoxy, aryl, —O-aryl, and (CO)OR₄, wherein R₄ is H or (C₁-C₄)alkyl and pharmaceutically acceptable salts thereof. In one embodiment, X is O or S, R₁ is H and R₂, and R₃ are independently selected from the group consisting of H, halo, (C₁-C₄)alkyl, (C₁-C₄)alkoxy, phenyl, —O-phenyl and (CO)OR_{4,} wherein R₄ is (C₁-C₄)alkyl.

In another embodiment, the compounds has the general structure of Formula I, wherein X is O or S, R₁ is H and R₂, and R₃ are independently selected from the group consisting of H, Cl, (C₁-C₂)alkyl, (C₁-C₂)alkoxy, phenyl, —O-phenyl and (CO)OCH₂CH₃ and pharmaceutically acceptable salts thereof. In one embodiment X is O, R₁ is H, R₂ is selected from the group consisting of H, Cl, (C₁-C₄)alkyl, (C₁-C₄)alkoxy, phenyl, —O-phenyl, and (CO)OR_{4,} and R₃ is selected from the group consisting of H, (C₁-C₄)alkyl, (C₁-C₄)alkoxy, phenyl, —O-phenyl and (CO)OR_{4,} wherein R₄ is (C₁-C₄)alkyl.

In one embodiment, a subject in need of treatment with a compound of the invention is treated with a compound of the invention selected from the group consisting of compounds 16 to 30:

4-(4′-Phenoxyphenyl)-thiazol-2-yl ammonium iodide (16); 4-[4′-(4-Chlorophenoxy)-phenyl]-thiazol-2-yl ammonium iodide (17); 4-[4-(3′-Chlorophenoxy)-phenyl]-thiazol-2-yl ammonium iodide (18); 4-[4-(2′-Chlorophenoxy)-phenyl]-thiazol-2-yl ammonium iodide (19); 4-[4′-(3,4-Dichlorophenoxy)-phenyl]-thiazol-2-yl ammonium iodide (20); 4-[4′-(4-Methoxyphenoxy)-phenyl]-thiazol-2-yl ammonium iodide (21); 4-[4′-(p-Toluoxy)phenyl]-thiazol-2-yl ammonium iodide (22); 4-[4′-(Biphenyl-4-yloxy)-phenyl]-thiazol-2-yl ammonium iodide (23); 4-[4′-(4-Phenoxy-phenoxy)-phenyl]-thiazol-2-yl ammonium iodide (24); 4-[4-(3′-Ethoxycarbonyl-phenoxy)-phenyl]-thiazol-2-yl ammonium iodide (25); 4-(4′-Phenylsulfanyl-phenyl)-thiazol-2-yl ammonium iodide (26); 4-[4-(4′-Chloro-phenylsulfanyl)-phenyl]-thiazol-2-yl ammonium iodide (27); 4-[4-(3′,4′-Dichloro-phenylsulfanyl)-phenyl]-thiazol-2-yl ammonium iodide (28); 4-[4-(4′-Methoxy-phenylsulfanyl)-phenyl]-thiazol-2-yl ammonium iodide (29); and 4-(4′p-Tolylsulfanyl-phenyl)-thiazol-2-yl ammonium iodide (30), or analogs, derivatives, and modifications thereof. In one aspect, the subject in need of treatment with a compound of the invention is selected from the group consisting of subjects having ER positive, ER negative, and adriamycin-resistant breast cancer cells.

In general, methods for the identification of a compound of the invention which has anti-cancer activity, i.e., anti-proliferative activity or anti-tumor activity, include the following general steps:

The test compound is adiministered to a cell, tissue, sample, or subject, in which the measurements are to be taken. A control is a cell, tissue, sample, or subject in which the test compound has not been added. A higher or lower level of the indicator or parameter being tested, i.e., cell proliferation, tumor growth, etc., in the presence of the test compound, compared with the levels of the indicator or parameter in the sample which was not treated with the test compound, is an indication that the test compound has an effect on the indicator or parameter being measured, and as such, is a candidate for inhibition of the desired activity. Various assays are known in the art to measure cell proliferation and tumor growth, both in vitro and in vivo. Some assays are direct measures of cell proliferation and tumor growth, while other assays may measure cell proliferation and tumor growth indirectly. Test compounds may be added at varying doses and frequencies to determine the effective amount of the compound which should be used and effective intervals in which it should be administered. In another aspect, an analog, derivative, or modification of the test compound may be used.

In accordance with one embodiment, the compounds of the present invention, and modifications, analogs, and derivatives thereof, can be formulated as pharmaceutical compositions by combining the compounds with one or more pharmaceutically acceptable carriers, fillers, solubilizing agents and stabilizers known to those skilled in the art. Such pharmaceutical compositions can be utilized to treat patients with proliferative disorders in need of such treatment.

Pharmaceutical compositions comprising the compounds of the present invention are administered to an individual in need thereof by any number of routes including, but not limited to, topical, oral, intravenous, intramuscular, intra-arterial, intramedullary, intrathecal, intraventricular, transdermal, subcutaneous, intraperitoneal, intrasal, enteral, topical, sublingual, or rectal means, with oral and intravenous routes being preferred. When administered orally, the compounds can be administered as a liquid solution, powder (Oyophilized or otherwise), tablet, capsule, or lozenge. Furthermore, oral formulations may include one or more of the present compounds in combination with one or more conventional pharmaceutical additive or excipients that are typically used in the preparation of tablets, capsules, lozenges, and other orally administrable forms. When administered as an intravenous solution, the derivatives of the present invention can be admixed with conventional intravenous solutions to form injectable aqueous or oily suspensions or solutions.

In accordance with one embodiment, a compound of the present invention is combined with other known anti-proliferative or anti-cancer agents to enhance the performance of such compounds and decrease the incidence of negative side effects. For example, compositions according to the present invention may comprise a compound of the present invention, or an analog, modification, and derivative thereof and their pharmaceutically acceptable salts, combined in a single pharmaceutical composition for simultaneous administration, or presented separately for administration in close succession.

The invention relates to administration of an identified compound in a pharmaceutical composition to practice the methods of the invention, the composition comprising the compound or an appropriate derivative or fragment of the compound and a pharmaceutically-acceptable carrier. As used herein, the term “pharmaceutically-acceptable carrier” means a chemical composition with which an appropriate compound of the invention may be combined and which, following the combination, can be used to administer the appropriate compound to a subject.

In one embodiment, the pharmaceutical compositions useful for practicing the invention may be administered to deliver a dose of between 1 ng/kg/day and 100 mg/kg/day.

Other pharmaceutically acceptable carriers which are useful include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3 butane diol, for example. Other acceptable diluents and solvents include, but are not limited to, Ringer's solution, isotonic sodium chloride solution, and fixed oils such as synthetic mono- or di-glycerides.

Pharmaceutical compositions that are useful in the methods of the invention may be administered, prepared, packaged, and/or sold in formulations suitable for oral, rectal, vaginal, parenteral, topical, pulmonary, intranasal, buccal, ophthalmic, or another route of administration. Other contemplated formulations include projected nanoparticles, liposomal preparations, resealed erythrocytes containing the active ingredient, and immunologically-based formulations.

The compositions of the invention may be administered via numerous routes, including, but not limited to, oral, rectal, vaginal, parenteral, topical, pulnonary, intranasal, buccal, or ophthalmic administration routes. The route(s) of administration will be readily apparent to the skilled artisan and will depend upon any number of factors including the type and severity of the disease being treated, the type and age of the veterinary or human patient being treated, and the like.

Different methods and formulations are known in the art for administration of therapeutic and cancer chemotherapeutic agents.

Pharmaceutical compositions that are useful in the methods of the invention may be administered systemically in oral solid formulations, ophthalmic, suppository, aerosol, topical or other similar formulations. In addition to the compound such as heparan sulfate, or a biological equivalent thereof, such pharmaceutical compositions may contain pharmaceutically-acceptable carriers and other ingredients known to enhance and facilitate drug administration. Other possible formulations, such as nanoparticles, liposomes, resealed erythrocytes, and immunologically based systems may also be used to administer, for example, substituted 4-aryloxy-and 4-arylsulfanyl-phenyl-2-aminothiazoles derivatives according to the methods of the invention. The method should not be construed to be limited to the general structure I, but should be construed to include other derivatives thereof.

The active ingredient may be present in the pharmaceutical composition in the form of a physiologically acceptable ester or salt, such as in combination with a physiologically acceptable cation or anion, as is well known in the art.

Compounds which are identified or prepared using any of the methods described herein may be formulated and administered to a mammal as described herein. Methods for identifying compounds with cancer therapeutic or anti-proliferative activity are known in the art.

The invention also includes a kit comprising a composition of the invention and an instructional material which describes administering the composition to a cell or to a tissue of a mammal. In another embodiment, this kit comprises a (preferably sterile) solvent suitable for dissolving or suspending the composition of the invention prior to administering the compound to the mammal.

The present invention also provides a pharmaceutical pack or kit comprising one or more containers containing one or more of the compounds of the present invention, or analogs, modifications, and derivatives thereof. In accordance with one embodiment, a kit is provided for a subject with cancer. In one aspect, the subject is a human. In this embodiment, the kit may comprise one or more compounds of the present invention, as well as other known anti-cancer agents. These pharmaceuticals can be packaged in a variety of containers, e.g., vials, tubes, microtiter well plates, bottles, and the like. Preferably, the kits will also include instructional materials.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the inructional material may describe one or more methods of alleviation the diseases or disorders in a cell or a tissue of a mammal. The instructional material of the kit of the invention may, for example, be affixed to a container which contains a compound of the invention or a composition comprising a compound of the invention, or be shipped together with a container which contains the peptide. Alternatively, the instructional material may be shipped separately from the container with the intention that the instructional material and the compound be used cooperatively by the recipient

In one embodiment, a composition comprising a compounds of the present invention, or analogs, modifications, and derivatives thereof, is used as a therapeutic agent in mammalian subjects, including both human and domesticated animals. More particularly, compositions comprising the present substituted 4-aryloxy-and 4-arylsulfanyl-phenyl-2-aminothiazoles derivatives are administered either orally or parenterally to a mammalian species to inhibit cancer cell proliferation and tumor growth. In another embodiment, compositions comprising one or more of the present substituted 4-aryloxy-and 4-arylsulfanyl-phenyl-2-aminothiazoles derivatives are administered either orally or parenterally to a mammalian species to inhibit tumor growth. When administered orally, the compounds are administered as a liquid solution, powder, tablet, capsule, or lozenge. The compounds can be used in combination with one or more conventional pharmaceutical additives or excipients used in the preparation of tablets, capsules, lozenges and other orally administrable forms. When administered parenterally, and more preferably by intravenous injection, the derivatives of the present invention can be admixed with saline solutions and/or conventional intravenous solutions. Other administration methods may be used and are described herein or are known to those of skill in the art.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, and mammals, including commercially relevant mammals such as cattle, pigs, horses, sheep, cats, and dogs.

Pharmaceutical compositions that are useful in the methods of the invention may be prepared, packaged, or sold in formulations suitable for oral, rectal, vaginal, parenteral, topical, pulmonary, intranasal, buccal, ophthalmic, intrathecal or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers such as cyanide and cyanate scavengers.

Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture.

Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide for pharmaceutically elegant and palatable preparation. Hard capsules comprising the active ingredient may be made using a physiologically degradable composition, such as gelatin Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose.

Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Methods for impregnating or coating a material with a chemical composition are known in the art, and include, but are not limited to methods of depositing or binding a chemical composition onto a surface, methods of incorporating a chemical composition into the structure of a material during the synthesis of the material (i. e., such as with a physiologically degradable material), and methods of absorbing an aqueous or oily solution or suspension into an absorbent material, with or without subsequent drying.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as cream-s, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulnonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention. Another formulation suitable for intranasal administration is a coarse powder comprising the active ingredient and having an average particle from about 0.2 to 500 micrometers. Such a formulation is administered in the manner in which snuff is taken, i.e., by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1-1.0% (w/w) solution or suspension of the active ingredient in an aqueous or oily liquid carrier. Such drops may further comprise buffering agents, salts, or one or more other of the additional ingredients described herein. Other ophthalmically-administrable formulations which are useful include those which comprise the active ingredient in microcrystalline form or in a liposomal preparation.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

Typically, dosages of the compound of the invention which may be administered to a subject, preferably a human, will vary depending upon any number of factors, including but not limited to, the type of subject, the type of surgery or procedure being performed on the subject, the disease state being treated, the age of the subject and the route of administration.

A compound of the invention can be administered to a subject as frequently as several times per hour, or it may be administered more or less frequently. The frequency of the dose will be readily apparent to the skilled artisan and will depend upon any number of factors, such as, but not limited to, the route of administration, the severity of the disease being treated, the type and age of the subject, and the type of surgery or procedure being performed on the subject, etc.

In accordance with one embodiment, a method is provided for treating a cancer in a human subject. The method comprises the steps of administering to the subject a composition comprising a compound represented by a formula which comprises the structure of formula I. In one aspect, a compound of the invention is selected from the group consisting of compounds 16 to 30, or analogs, derivatives, and modifications thereof.

All references discussed herein are incorporated by reference. One skilled in the art will readily appreciate that the present invention is well adapted to carry out the objects and obtain the ends and advantages mentioned, as well as those inherent therein. The present invention may be embodied in other specific forms without departing from the spirit or essential attributes thereof and, accordingly, reference should be made, to the appended claims, rather than to the foregoing specification, as indicating the scope of the invention.

Compounds of the invention may be prepared according to the synthetic schemes provided herein.

The invention is now described with reference to the following examples. These examples are provided for the purpose of illustration only and the invention should in no way be construed as being limited to these examples but rather should be construed to encompass any and all variations which become evident as a result of the teaching provided herein.

In accordance with the present invention, as described above or as discussed in the Examples below, there can be employed conventional clinical, chemical, cellular, histochemical, biochemical, molecular biology, microbiology and recombinant DNA techniques which are known to those of skill in the art. Such techniques are explained fully in the literature.

The invention should not be construed to be limited solely to the assays and methods described herein, but should be construed to include other methods and assays as well. One of skill in the art will know that other assays and methods are available to perform the procedures described herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Experimental Examples

The invention is now described with reference to the following Examples. These Examples are provided for the purpose of illustration only and the invention should in no way be construed as being limited to these Examples, but rather should be construed to encompass any and all variations which become evident as a result of the teaching provided herein.

### EXAMPLES

### Materials and Methods

All starting materials were purchased from Aldrich and were used as received. Melting points were measured on an Electrothermal Mel-Temp and are uncorrected. Carbon and proton NMR spectra were recorded on a General Electric 300 MHz spectrometer. Mass spectra were obtained on a Finnagan LcQ Classic spectrometer. High resolution (El) mass spectra were obtained from the University of Illinois, Urbana-Champaign. Combustion analyses were performed by Atlantic Microlabs Inc.

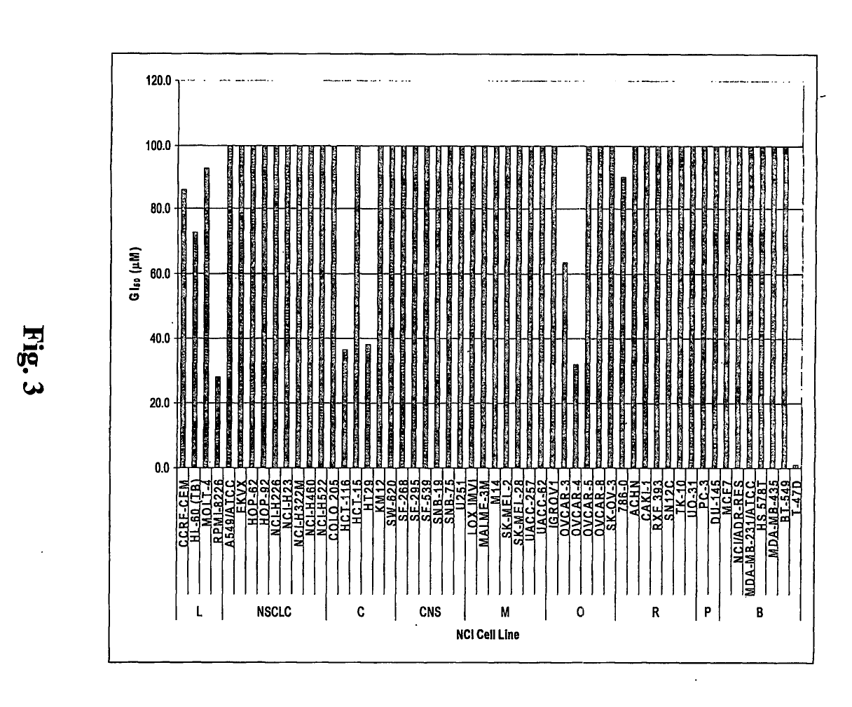

### General Procedure for Synthesis of 4-Aryloxy and 4-Arylsulfanyl Substituted Acetophenones (compounds 2-15; see Scheme I and FIG. 3).

Anhydrous K₂CO₃ (12 mmol) was added to a solution of 4′-fluoroacetophenone (10 mmol) and the corresponding phenol or thiol (10 mmol) in N,N-dimethylacetamide (DMAC, 10 mL). The suspension was refluxed for 8-10 h, cooled to room temperature, and diluted with H₂O (10 mL). In some instances (compounds 8 and 9), the addition of H₂O resulted in the deposition of the product as a solid which was collected by filtration. In those instances where the product was not a solid, the resulting solution was extracted with CHCl₃ (3×15 mL), dried over MgSO₄, and concentrated in vacuo to yield a brown oil. The remaining DMAC was removed by Kugelrohr distillation. The viscous oil was allowed to cool and solidify. The crude solid was recrystallized from EtOH (see Yeager and Schissel, Synthesis, 1991, 1:63-68).

4-(4′-Chlorophenoxy)-acetophenone (Compound 2).

Isolated as a light brown solid (1.81 g, 73%); mp 65-66° C. (lit.¹²66-68° C.); ¹H NMR (CDCl₃) δ 2.57 (s, 3H), 6.99 (d, J=6.4 Hz, 4H), 7.35 (d, J=8.3 Hz, 2 H), 7.94 (d, J=8.2 Hz, 2H); ¹³C NMR δ 27.0, 117.8, 121.7, 130.2, 130.6, 131.2, 132.8, 154.7, 162.0, 197.1; MS APCI m/z 247 (M)⁺.

4-(3′-Chlorophenoxy)-acetophenone (Compound 3).

Isolated as an orange oil (2.03 g, 82%); ¹H NMR (CDCl₃) δ 2.49 (s, 3H), 6.82-7.13 (m, 5H), 7.22 (t, J=7.9 Hz, 1H), 7.88 (d, J=7.7 Hz, 2H); ¹³C NMR δ26.9, 109.9, 118.3, 118.5, 120.6, 125.0, 131.1, 133.0, 135.7, 157.0, 161.4, 196.9; MS APCI m/z 247 (M)⁺.

4-(2′-Chlorophenoxy)-acetophenone (Compound 4).

Isolated as a yellow solid (1.64 g, 67%); mp 41-43° C. (lit.¹⁷ 49-50° C.); ¹H NMR (CDCl₃) δ 2.47 (s, 31), 6.85 (d, J=8.3 Hz, 21), 6.99-7.16 (n, 2H), 7.21 (d, J=7.5 Hz, 1H), 7.39 (d, J=7.5 Hz, 1H), 7.86 (d, J=8.3 Hz, 2H); ¹³C NMR δ 26.9, 109.9, 116.8, 123.0, 126.7, 127.2, 128.9, 131.1, 131.5, 132.5, 151.2, 161.7, 196.9; MS APCI m/z 247 (M)⁺.

4-(3′,4′-Dichlorophenoxy)-acetophenone (Compound 5).

Recrystallized from hexanes to afford a brown sold (2.61, 93%); mp 47-49° C. (lit¹⁸ 50-52° C.); 1H NMR (CDCl₃) δ 2.57 (s, 3H), 6.90 (d, J=8.4 Hz, 1H1), 7.01 (d, J=8.5 Hz, 2H), 7.14 (s, 1 H), 7.42 (d, J=8.6 Hz, 1H), 7.95 (d, J=8.3 Hz, 2I1); ¹³C NMR δ 27.0, 118.4, 119.7, 122.2, 128.5, 131.2, 131.8, 133.3, 134.0, 155.3, 161.2, 197.1; MS APCI m/z 281 (M)⁺.

4-(4′-Methoxyphenoxy)-acetophenone (Compound 6).

Isolated as a light brown solid (2.02 g, 84%); mp 56-58° C. (lit.¹⁹ 61° C.); ¹H NMR (CDCl₃) δ 2.55 (s, 31), 3.81 (s, 3H1), 6.90-7.00 (m, 6H), 7.90 (d, J=7.9 Hz, 2H); ¹³C NMRδ 26.9, 56.1, 115.6, 116.9, 122.2, 131.1, 131.9, 149.0, 157.2, 163.4, 197.2; MS APCI m/z243 (M)⁺.

4-(p-Toluoxy)-acetophenone (7).

Isolated as a light brown solid (0.91 g, 40%); mp 44-46° C.; ¹H NMR (CDCl₃) δ 2.37 (s, 3H), 2.57 (s, 31), 6.97 (d, J=7.5 Hz, 4H), 7.19 (d, J=7.7 Hz, 2H), 7.92 (d, J 8.5 Hz, 2H1); ¹³C NMR δ 21.3, 26.9, 117.4, 120.5, 120.7, 131.1, 132.1, 134.9, 153.5, 163.0, 197.2; MS APCI m/z 227 (M)⁺.

4-(Biphenyl4′-yloxy)-acetophenone (8).

Isolated as a pale yellow solid (2.61 g, 90%); mp 114-117° C.; ¹H NMR (CDCl₃) δ 2.59 (s, 3H), 7.06 (d, J=8.3 Hz, 21), 7.14 (d, J=8.1 Hz, 2H), 7.36 (d, J=8.0 Hz, 1H), 7.44 (t, J=7.4 Hz, 2H), 7.60 (t, J=7.1 Hz, 4H), 7.96 (d, J=8.0 Hz, 21); ¹³C NMR δ 27.0, 117.9, 120.9, 127.5, 127.8, 129.2, 129.4, 131.1, 132.5, 138.2, 140.8, 155.5, 162.4, 197.2; MS APCI m/z 289 (M)⁺.

4-(4′-Phenoxy-Phenoxy)-acetophenone (9).

Isolated as a white solid (2.47 g, 81%); mp 75-77° C.; 1H NMR (CDCl₃) δ 2.57 (s, 3H), 6.80-7.40 (m, 1H), 7.94 (d, J=8.1 Hz, 2H); ¹³C NMR δ 26.9, 117.3, 119.2, 120.9, 122.1, 123.9, 130.3, 131.1, 132.3, 151.3, 154.5, 157.8, 162.9, 197.2; MS APCI m/z 305 (M)⁺.

Ethyl 3-(4′-Acetyl-Phenoxy)-Benzoate (10).

Isolated as a yellow oil (1.92 g, 68%); ¹H NMR (CDCl₃) δ 1.37 (t, J=6.7 Hz, 3H), 2.56 (s, 3H), 4.35 (q, J=7.1 Hz, 3H), 6.98 (d, J=8.1 Hz, 2H), 7.24 (d, J=7.9 Hz, 1H), 7.44 (t, J=7.5 Hz, 1H), 7.71 (s, 1H), 7.86 (d,J=7.5 Hz, 1H), 7.93 (d,J=8.1 Hz, 3H); ¹³C NMR δ 14.8, 26.9, 61.8, 118.0, 121.5, 125.0, 126.1, 130.5, 131.2, 132.8, 133.2, 156.1, 161.9, 166.2, 197.1; MS APCI m/z 285 (M)⁺.

(4′-Phenylsulfanyl)-acetophenone (11).

Isolated as an orange solid (0.87 g, 38%); mp 59-61° C. (lit.²⁰ 67-68° C.); ¹H NMR (CDCl₃) δ 2.55 (s, 3H), 7.21 (d,J=8.1 Hz, 2H), 7.40-7.47 (m, 5H), 7.82 (d, J=8.1 Hz, 2H); ¹³C NMR δ 13.7, 128.0, 129.3, 129.4, 130.2, 132.6, 134.4, 135.0, 145.4, 192.9; MS APCI m/z 229 (M)⁺.

4-(4′-Chloro-Phenylsulfanyl)-acetophenone (12).

Isolated as an orange solid (1.68 g, 64%); mp 40-42° C.; ¹H NMR (CDCl₃) 32.56 (s, 3H), 7.22 (d,J=8.1 Hz, 2H), 7.38 (d, J=6.5 Hz, 4H), 7.83 (d, J=7.9 Hz, 2H1); ¹³C NMR δ 27.0, 128.3, 129.5, 130.4, 131.4, 135.4, 143.7, 144.5, 147.8, 197.5; MS APCI m/z 263 (M)⁺.

4-(3′,4′-Dichloro-Phenylsulfanyl)-acetophenone (13).

Isolated as a red oil (1.44 g, 87%); 1H NMR (CDCl₃) δ 2.56 (s, 3H), 7.23-7.33 (m, 2H), 7.39-7.53 (m, 1H), 7.85 (d,J=8.1 Hz, 4H); ¹³C NMR δ 27.0, 129.4, 129.7, 131.6, 131.8, 132.3, 133.6, 133.9, 134.0, 134.5, 136.0, 197.5; MS APCI m/z 297 (M)⁺.

4-(4′-Methoxy-phenylsulfanyl)-acetophenone (14).

Isolated as a red solid (1.49 g, 58%); mp 25-27° C.; ¹H NMR (CDCl₃) δ 2.51 (s, 3H), 3.83 (s, 3H), 6.94 (d, J=8.1 Hz, 2H), 7.07 (d,J=8.1 Hz, 2H), 7.45 (d,J=8.1 Hz, 2), 7.76 (d,J=8.1 Hz, 2H); 3C NMR 26.9, 55.9, 115.9, 121.8, 126.3, 129.3, 134.4, 137.3, 147.4, 161.2, 197.6; MS APCI m/z 259 (M)⁺.

4-(4′-Tolylsulfanyl)-acetophenone (15).

Isolated as a red solid (1.64 g, 68%); mp 90-92° C.; H NMR (CDCl₃) δ 2.40 (s, 3H), 2.54 (s, 3H), 7.15 (d, J=8.1 Hz, 2H), 7.22 (d, J=7.7 Hz, 2H), 7.41 (d, J=7.5 Hz, 2H), 7.79 (d, J=8.0 Hz, 2H); ¹³C NMR δ 21.8, 27.0, 127.2, 128.4, 129.3, 131.0, 134.6, 135.0, 140.0, 146.4, 197.6; MS APCI m/z 243 (M)⁺.

See the following references for details of the various techniques described above: Yeager and Schissel, Synthesis, 1991, 1:63-68; Chordia et al., Bioorg. Med. Chem. Lett., 2002, 12:12:1563-1566; Monks et al., J. Natl. Cancer Inst., 1991, 83:757-766; Gray and Wickstrom, Biotechniques, 1996, 21:5:780-782; Tinley et al., Cancer Res., 2003, 63:3211-3220; Kimoto et al., J. Pharm. Soc. Japan, 1954, 74:358-360; Chem. Abs. 1955, 5373; Markley et al., J. Med. Chem., 1986, 29:3:427-433; Petit and Buu-Hoï, J. Org. Chem., 1961,26:3832-3834; and Szrnant and Palopoli, J. Amer. Chem. Soc., 1950, 72:1757-1758.

### General Procedure for Synthesis of Substituted 4-aryloxy and 4-arylsulfanyl-phenyl-2-aminothiazole Salts (Compounds 16-30).

Thiourea (40 mmol) and iodine (11 mmol) were added to a stirring solution of the appropriate acetophenone (10 mmol) in absolute ethanol (20 mL). The mixture was heated at 100° C. for 2-3 h in an open vessel. The crude residue was washed with ether (3×50 mL) and was recrystallized from hot water. A few of these compounds (16, 17, and 26) in free amine or HCl salt form are reported in the literature (see Hargrave et al., J. Med. Chem., 1983, 26:8:1158-1163; Kawamatsu et al., Eur. J. Med. Chem. Chim. Ther., 1981, 16:4:355-362; Chordia et al., Bioorg. Med. Chem. Lett., 2002, 12:12:1563-1566; and Scheme I herein).

4-(4′-Phenoxyphenyl)-thiazol-2-yl Ammonium Iodide (compound 16).

Isolated as a yellow solid (1.82 g, 46%); mp 193-195° C.; ¹H NMR (DMSO-d⁶) δ 7.02 (t, J=8 Hz, 4H), 7.08 (s, 1H), 7.13 (t, J=6.2 Hz, 1H1), 7.37 (t, J=7.7 Hz, 2H), 7.65 (d,J=8.1 Hz, 2H); ¹³C NMR δ 103.0, 119.5, 120.1, 125.1, 128.8, 131.2, 140.1, 145.7, 156.4, 158.6, 171.1; MS APCI m/z 269 (M-HI)⁺; Anal. calcd. for C₁₅H₁₃IN₂OS: C, 45.47; H, 3.31; N, 7.07. Found: C, 45.25; H, 3.26; N, 7.08.

4-[4′-(4-Chlorophenoxy)-phenyl]-thiazol-2-yl Ammonium Iodide (Compound 17).

Isolated as a light orange solid (0.31 g, 71%); mp 152-154° C.; ¹H NMR (DMSO-d⁶) δ 6.95-7.10 (m, 5H), 7.41 (t, J=6.6 Hz, 2H), 7.67 (d, J=8.7 Hz, 1H), 7.92 (d, J=8.7 Hz, 1H), 8.94 (br s); 1³C NMR δ 109.8, 118.6, 118.8, 121.9, 123.1, 128.9, 131.0, 131.2, 131.7, 155.6, 170.0; HRMS calcd for C₁₅H₁₂ClIN₂OS (M-HI)⁺m/z 303.036100, found m/z 303.035888.

4-[4-(3′-Chlorophenoxy)-phenyl]-thiazol-2-yl Ammonium Iodide (Compound 18).

Isolated as a yellow solid (0.57 g, 65%); mp 107-110° C.; ¹H NMR (DMSO-d⁶) δ 6.96 (d, J=7.9 Hz, 1H), 7.11 (s, 1H), 7.18 (d, J=8.1 Hz, 1H), 7.39 (t, J=7.5 Hz, 11H), 7.70 (d, J=8.2 Hz, 4H), 7.93 (d, J=7.8 Hz, 1H), 8.96 (br s); ¹³C NMR δ 103.2, 113.9, 118.4, 119.7, 120.2, 124.8, 128.9, 132.6, 135.0, 144.1, 157.6, 160.6, 171.0; HRMS calcd for C₁₅H₁₂ClIN₂OS (M-HI)⁺m/z 303.036700, found m/z 303.035888; Anal. calcd. for C₁₅H₁₂ClIN₂OS·0.6 H₂O: C, 40.81; H, 2.72; N, 6.34. Found: C, 40.53; H, 2.60; N, 6.10.

4-[4-(2′-Chlorophenoxy)-phenyl]-thiazol-2-yl Ammonium Iodide (Compound 19).

Isolated as a yellow solid (0.35 g, 40%); mp 160-163° C.; ¹H NMR (DMSO-d⁶) δ 6.98 (d, J=8.3 Hz, 2H), 7.08 (s, 1H), 7.17 (d, J=8.1 Hz, 1H), 7.23 (t, J=6.6 Hz, 1H), 7.37 (t, J=7.7 Hz, 1H), 7.58 (d, J=7.6 Hz, 1H), 7.68 (d, J=8.3 Hz, 2H), 8.99 (br s); ¹³C NMR δ 103.0, 118.2, 123.0, 126.9, 127.2, 128.9, 130.0, 131.7, 131.8, 133.4, 147.5, 158.3, 171.0; MS APCI m/z 303 (M-HI)⁺; Anal. calcd. for C15H₁₂ClIN2OS: C, 41.83; H, 2.81; N, 6.50. Found: C, 41.65; H, 2.71; N, 6.48.

4-[4′-(3,4-Dichlorophenoxy)-phenyl]-thiazol-2-yl Ammonium Iodide (Compound 20).

Isolated as a brown solid (0.35 g, 75%); mp 202-205° C.; ¹H NMR (DMSO-d⁶) δ 7.07 (d, J=7.5 Hz, 3H), 7.14 (s, 1H), 7.37 (s, 1H), 7.62 (d, J- 8.2 Hz, 1H), 7.70 (d, J=8.1 Hz, 1H), 7.93 (d, J=8.1 Hz, 1H), 9.02 (br s); ¹³C NMR δ 103.4, 118.9, 120.2, 120.9, 121.7, 122.6, 129.0, 131.7, 132.7, 133.2, 133.5, 161.0, 171.1; HRMS calcd for C₁₅H₁₁Cl₂IN₂OS (M-HI)⁺ m/z 336.997000, found m/z 336.996915; Anal. calcd. for C₁₅H₁₁Cl₂IN₂OS·0.9 H₂O: C, 37.43; H, 2.28; N, 5.82. Found: C, 37.19; H, 2.25; N, 5.66.

4-[4′-(4-Methoxyphenoxy)-phenyl]-thiazol-2-yl Ammonium Iodide (Compound 21).

Isolated as a yellow solid (0.37 g, 87%); mp 204-207° C.; ¹H NMR (DMSO-d⁶) δ 3.71 (s, 3H), 6.95-7.03 (m, 5H), 7.05 (s, 1H), 7.62 (d, J=8.2 Hz, 2H), 7.98 (d,J=7.9 Hz, 1H), 8.93 (brs); ¹³C NMR δ 56.4, 102.6, 116.2, 117.1, 118.2, 122.1, 122.6, 124.5, 128.7, 131.6, 150.0, 171.1; MS APCI m/z 299 (M-HI)⁺; Anal. calcd. for C₁₆H₁₅IN₂O₂S: C, 45.08; H, 3.55; N, 6.57. Found: C, 44.97; H, 3.65; N, 6.67.

4-[4′-(p-Toluoxy)phenyl]-thiazol-2-yl Ammonium Iodide (Compound 22).

Isolated as a light brown solid (0.27 g, 65%); mp 118-120° C.; ¹H NMR (DMSO-d⁶) δ 2.26 (s, 3H), 6.90-7.03 (m, 5H), 7.19 (t,J=7.9 Hz, 2H), 7.65 (d,J=8.6 Hz, 1H), 7.90(d,J=6.7Hz, 1H); ¹³C NMR 627.5, 117.6, 118.9, 120.3, 121.0, 128.6, 131.5, 131.6, 134.2, 134.9, 161.2, 162.6; MS APCI m/z 283 (M-HI)⁺; Anal. calcd. for C₁₆H₁₅IN₂OS: C, 46.84; H. 3.69, N, 6.83. Found: C, 46.65; H, 3.86; N, 6.61.

4-[4′-(Biphenyl4-yloxy)-phenyl]-thiazol-2-yl Ammonium Iodide (Compound 23).

Isolated as a yellow solid (0.33 g, 70%); mp 253-254° C.; ¹H NMR (DMSO-d⁶) 7.00-7.20 (m, 4H), 7.30 (d, J=6.5 Hz, 1H), 7.40 (t, J=7.3 Hz, 2H), 7.66 (m, 6H), 7.93 (d,J=8.0 Hz, 1H); ¹³C NMR δ 102.8, 119.8, 120.4, 127.4, 128.2, 128.8, 129.4, 129.5, 129.9, 136.7, 137.3, 156.4, 158.2, 159.4, 177.5; HRMS calcd for C₂₁H₁₇IN₂OS (M-HI)⁺ m/z 345.106200, found m/z 345.106160; Anal. calcd. for C₂₁H₁₇IN₂OS: C, 53.40; H, 3.63; N, 5.93. Found. C, 53.65; H, 3.65; N, 5.80.

4-[4′-(4-Phenoxy-phenoxy-phenyl]-thiazol-2-yl ammonium iodide (Compound 24).

Isolated as a pale yellow solid (0.42 g, 86%); mp 137-140° C.; ¹H NMR (DMSO-d⁶) 6.95-7.12 (m, 10H), 7.34 (t, J=7.4 Hz, 2H), 7.67 (d, J=8.1 Hz, 1H), 7.92 (d, J=8.3 Hz, 1H); 13C NMR δ 102.8, 117.7, 119.2, 119.3, 121.4, 122.0, 122.7, 124.3, 128.8, 131.0, 131.7, 143.9, 152.3, 153.3, 161.1; HRMS calcd for C₂H₁₇IN₂O₂S (M-HI)⁺ m/z 361.101000, found m/z 361.101075. 4-[4-(3′-Ethoxycarbonyl-phenoxy)-phenyl]-thiazol-2-yl ammonium iodide (compound 25). Isolated as a yellow solid (1.01 g, 64%); mp 158-160° C.; ¹H NMR (DMSO-d⁶) δ 1.24 (t,J=6.3 Hz, 3H), 4.24 (q,J=7.0 Hz, 2H), 7.10 (s, 3H), 7.34 (d, J=7.9 Hz, 1H), 7.45 (s, 1H), 7.51 (t, J=7.7 Hz, 1H), 7.71 (d, J=7.2 Hz, 2H), 7.94 (d,J=7.9 Hz, 1H), 8.83 (brs); ¹³C NMR δ 15.0, 62.0, 103.2, 109.8, 118.7, 119.6, 120.1, 124.8, 125.5, 129.0, 131.8, 132.8, 157.3, 157.9, 165.9, 171.0; HRMS calcd. for C₁₈H₁₇IN₂O₃S (M-HI)⁺ m/z 341.095700, found m/z 341.095989; Anal. calcd. for C₁₈H₁₇IN₂O₃S·2 H₂O: C, 42.87; H, 3.37; N, 5.55. Found: C, 42.58; H, 3.26; N, 5.51.

4-(4′-Phenylsulfanyl-phenyl)-thiazol-2-yl ammonium iodide (compound 26).

Isolated as an orange solid (0.40 g, 98%); mp 129-132° C.; ¹H NMR (DMSO-d⁶) δ 7.15 (s, 1H), 7.26-7.47 (m, 6H), 7.65 (d, J=7.5 Hz, 2H), 7.81 (d, J=7.5 Hz, 1H); ¹³C NMR δ 104.0, 127.7, 128.4, 129.0, 130.0, 130.7, 131.1, 132.5, 164.0, 170.8, 177.5; HRMS calcd for C₁₅H₁₃IN₂S₂ (M-HI)⁺ m/z 285.052200, found m/z 285.052017.

4-[4-4′-Chloro-phenylsulfanyl)-phenyl]-thiazol-2-yl ammonium iodide (compound 27).

Isolated as an orange solid (0.44 g, 98%); mp 158-160° C.; ¹H NMR (DMSO-d⁶) 7.14 (s, 1H), 7.24-7.47 (m, 6H), 7.68 (d, J=7.9 Hz, 2H); ¹³C NMR δ 101.3, 110.1, 127.6, 127.9, 130.6, 132.0, 133.5, 143.6, 154.7, 167.8, 169.8; HRMS calcd for C₁₅H₁₂ClIN₂S₂ (M-HI)⁺ m/z 319.012900, found m/z 319.013045.

4-[4-(3′,4′-Dichloro-phenylsulfanyl)-phenyl]-thiazol-2-yl ammonium iodide (compound 28).

Isolated as a red solid (0.23 g, 48%); mp 45-47° C. (dec.); ¹H NMR (DMSO-d⁶) δ 7.18 (s, 1H), 7.24 (s, 2H), 7.46 (d, J=8.1 Hz, 2H), 7.59 (s, 1H), 7.69 (d,J=7.7Hz,2H); ¹³C NMR δ 104.8, 128.1, 131.2, 131.3, 132.2, 132.6, 132.8, 133.0, 133.2, 140.1, 150.3, 161.2, 171.1; HRMS calcd for C₁₅H₁₁Cl₂IN₂S₂ (M-HI)⁺ m/z 352.974100, found m/z 352.974073.

4-[4-(4′-Methoxy-phenylsulfanyl)-phenyl]-thiazol-2-yl ammonium iodide (compound 29).

Isolated as an orange solid (0.40 g, 91%); mp 202-205° C.; ¹H NMR (DMSO-d⁶) δ 3.74 (s, 3H), 6.98 (d, J=8.3 Hz, 21), 7.07 (s, 1H), 7.12 (d, J=7.9 Hz, 2H), 7.40 (d, J=8.1 Hz, 2H), 7.57 (d, J=7.7 Hz, 2H); ¹³C NMR δ 56.3, 103.4, 116.5, 116.7, 127.5, 128.4, 136.7, 140.3, 148.9, 154.3, 158.7, 169.7; MS APCI m/z 315 (M-HI)⁺; Anal. calcd. for C₁₆H₁₅IN₂OS₂: C, 43.44; H, 3.42; N, 6.33. Found: C, 43.18; H. 3.40; N, 6.32.

4-(4′p-Tolylsulfanyl-phenyl)-thiazol-2-yl ammonium iodide (30).

Isolated as an orange solid (0.23 g, 54%); mp 182-185° C.; ¹H NMR (DMSO-d⁶) δ 2.28 (s, 3H), 7.08 (s, 1H), 7.20 (m, 2H), 7.28 (d, J=7.5 Hz, 21), 7.62 (d, J=7.9 Hz, 4H); ¹³C NMR δ 21.2, 109.9, 127.6, 128.7, 129.2, 130.0, 131.4, 133.4, 140.5, 153.8, 156.5, 170.5; MS APCI m/z 299 (M-HI)⁺; Anal. calcd. for C16H15IN₂S₂: C, 45.07; H, 3.55; N, 6.57. Found: C, 44.86; H, 3.52; N, 6.23.

Testing Compounds of the Invention for growth-inhibitory activity.

NCI High Throughput Prescreen.

Each cell line was inoculated and preincubated on a microtiter plate. Test agents were then added at a single concentration and the culture incubated for 48 h. End-point determinations were made with alamar blue (Gray and Wickstrom, Biotechniques, 1996, 21:5:780-782). Results for each test agent were reported as the percent of growth of the treated cells when compared to the untreated control cells. Compounds which reduce the growth of any one of the cell lines to approximately 32% or less (negative numbers indicate cell kill) were passed on for evaluation in the full panel of 60 cell lines over a 5-log dose range.

NCI Anti-Tumor Screen.

Compounds were tested at the National Cancer Institute's Developmental Therapeutics Program, in duplicate, against 60 human tumor cell lines at a minimum of five concentrations at 10-fold dilutions: A 48 hour continuous drug exposure protocol was used, and a sulforhodamine B (SRB) protein assay was used to estimate cell viability or growth.

Inhibition of Breast Cell Proliferation.

MCF7 cells were treated with compounds or vehicle (DMSO). After a 48 hour incubation, the SRB assay was used to determine inhibition of proliferation and cytotoxicity (Skehan et al., J. Natl. Cancer Ins, 1990, 82:1107-1112). Percent inhibition of growth at 100 μM was determined and GI₅₀ values were calculated from log-dose response curves as previously described (Tinley et al., Cancer Res., 2003, 63:3211-3220).

### Results

The following synthetic scheme demonstrates a method of preparing compounds of the invention. Starting components, reagents, conditions, and intermediate steps and compounds are provided in Scheme I and in the Examples provided herein.

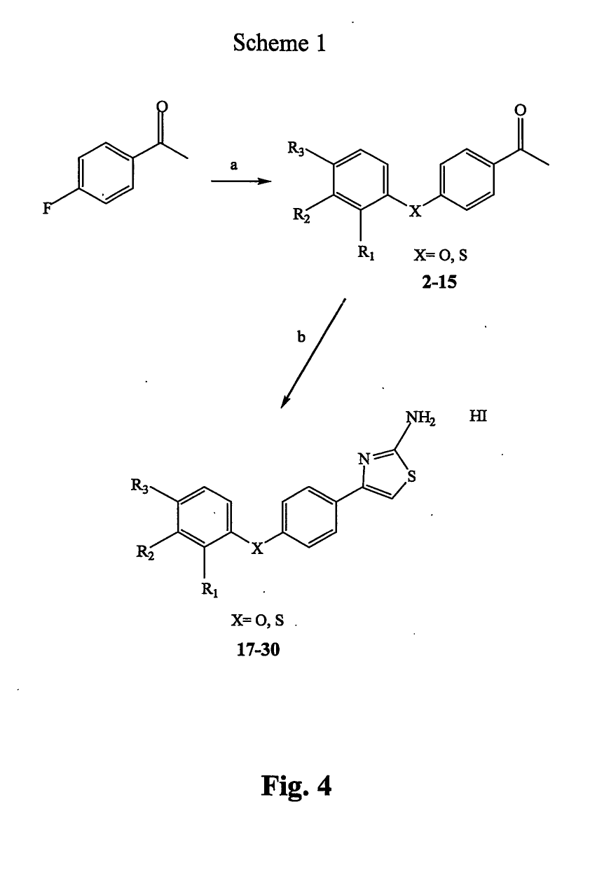

A general synthesis of the substituted 4-aryloxy- and 4-arylsulfanyl-phenyl-2-aminothiazole salts is shown in Scheme 1 (see also FIG. 4). The reagents and conditions utilized for Scheme 1 include: a) phenol or thiol, K₂CO₃, DMAC, reflux, 8-10 h, 38-93%; b) thiourea, I2, EtOH, 100° C, 3 h, 40-98%. Condensation of the appropriately substituted phenols with 4′-fluoroacetophenone afforded the corresponding 4-aryloxyacetophenones in 40-93% yield (Yeager and Schissel, Synthesis, 1991, 1:63-68). 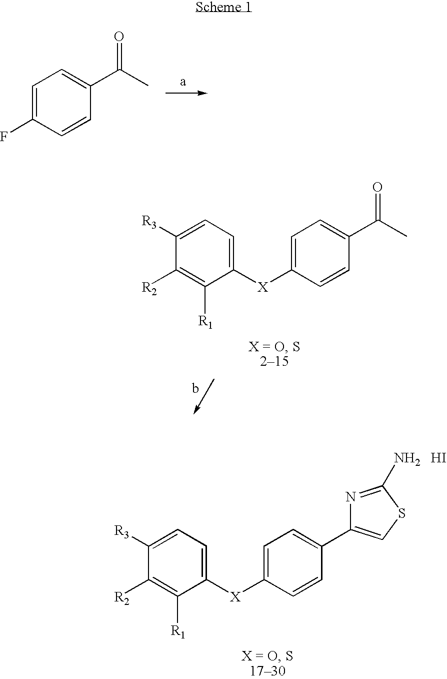
Reagents and conditions: a) phenol or thiol, K₂CO₃, DMAC, reflux, 8-10 h, 38-93%; b) thiourea, I_{2,} EtOH, 100° C., 3 h, 40-98%

This condensation reaction also proceeded smoothly with various substituted benzenethiols forming the 4-arylsulfanyl acetophenones in 38-87% yield. Treatment of the acetophenones with thiourea and iodine successfully generated the 2-aminothiazole salts (Table 1) in 40-98% yield (Chordia et al., Bioorg. Med. Chem. Lett, 2002, 12:12:1563-1566).

A general synthesis of the substituted 4-aryloxy- and 4-arylsulfanyl-phenyl-2-aminothiazaole salts is shown in Scheme 1 provided herein. Condensation of the appropriately substituted phenols with 4′-fluoroacetophenone afforded the corresponding 4-aryloxyacetophenones in 40-93% yield (Yeager and Schissel, Synthesis, 1991, 1:63-68). This condensation reaction also proceeded smoothly with various substituted benzenethiols forming the 4-arylsulfanyl acetophenones in 38-87% yield. Treatment of the acetophenones with thiourea and iodine successfully generated the 2-aminothiazole salts (Table 1) in 40-98% yield (Chordia et al., Bioorg. Med. Chem. Lett., 2002, 12:1563-1566) (see Table 1 and Scheme 1).

TABLE 1
Thiazoles

| Compound # | X | R₁ | R₂ | R₃ | % Yield |
|---|---|---|---|---|---|
| 17 | O | H | H | Cl | 71 |
| 18 | O | H | Cl | H | 65 |
| 19 | O | Cl | H | H | 40 |
| 20 | O | H | Cl | Cl | 75 |
| 21 | O | H | H | OMe | 87 |
| 22 | O | H | H | Me | 65 |
| 23 | O | H | H | Ph | 70 |
| 24 | O | H | H | OPh | 86 |
| 25 | O | H | CO₂Et | H | 64 |
| 26 | S | H | H | H | 98 |
| 27 | S | H | H | Cl | 98 |
| 28 | S | H | Cl | Cl | 48 |
| 29 | S | H | H | OMe | 91 |
| 30 | S | H | H | Me | 54 |

Upon completion of the synthesis, the thiazole compounds 17-24 and 27-30 were submitted to the NCI's antitumor screen (see Monks et al., J. Natl. Cancer Inst., 1991, 83:757b 766; Bray and Wickstrom, Biotechniques, 1996, 21:5:780-782) and the results against human breast cancer cell lines are shown in Table 2 as GI₅₀ values. Although not effective against other types of cancer, compound 17 (4-[4′-(4-Chlorophenoxy)-phenyl]-thiazol-2-yl ammonium iodide) showed selectivity for TA47D breast cancer cells with a GI₅₀ of 0.917 μM (FIG. 3).

Compounds 20 (4-[4′-(3,4-Dichlorophenoxy)-phenyl]-thiazol-2-yl ammonium iodide) and 28 (4-(4-[4-(3′,4′-Dichloro-phenylsulfanyl)-phenyl]-thiazol-1-yl ammonium iodide) showed selectivity for the adriamycin-resistant cell line with GI₅₀ values of 1.29 μM and 3.37 μM respectively. Adriamycin-resistant selectivity is clinically important because patients develop cancer that is resistant to treatment.

TABLE 2
Human Breast Cancer Cytotoxicity Data.

| | % | | | | | | | |
|---|---|---|---|---|---|---|---|---|
| | Inhibition | | | | | | | |
| | of | GI₅₀ (μM) | | | | | | |
| | MCF7 | | NCI/ | MDA- | | MDA- | | |
| | cells | | ADR- | MB- | HS | MB- | BT- | T- |
| Compound # | at 100 μM | MCF7 | RES | 231/ATCC | 578T | 435 | 549 | 47D |
| 16 | 81 | ND | ND | ND | ND | ND | ND | ND |
| 17 | 85 | >100 | >100 | >100 | >100 | >100 | >100 | 0.917 |
| 18 | 100 | 14.6 | 13.2 | 4.80 | 19.7 | 2.10 | 14.3 | 14.0 |
| 19 | 87 | 27.4 | 25.8 | 10.5 | ND | 14.8 | 12.8 | 16.4 |
| 20 | 47 | 3.02 | 1.29 | 2.29 | 2.00 | 0.759 | 1.91 | 20.9 |
| 21 | 24 | ND | ND | ND | ND | ND | ND | ND |
| 22 | 98 | 17.2 | 18.2 | 14.1 | 22.7 | 11.8 | ND | 0.54 |
| 23 | 53 | 14.3 | 17.4 | 12.6 | 18.8 | 18.6 | 10.4 | 3.91 |
| 24 | 81 | 35.1 | 35.9 | 12.6 | 27.8 | 22.7 | 16.1 | 4.9 |
| 25 | 84 | ND | ND | ND | ND | ND | ND | ND |
| 26 | 79 | ND | ND | ND | ND | ND | ND | ND |
| 27 | 89 | 21.6 | 17.5 | 12.4 | 18.8 | 1.49 | 15.3 | 24.4 |
| 28 | 54 | 5.68 | 3.37 | 3.73 | ND | 1.11 | 5.95 | 2.79 |
| 29 | 11 | ND | ND | ND | ND | ND | ND | ND |
| 30 | 96 | 25.9 | 64.6 | >100 | 59.6 | 1.4 | 25.7 | 68 |

Interestingly, when the GI₅₀ values of compounds 17 (X═O) and 27 (X═S) are compared, there appears to be a relationship between heteroatom substitution and selectivity for the estrogen receptor-positive or estrogen receptor-negative cell lines. The GI₅₀ of 17 exceeded 100 μM in all cell lines except for the estrogen-positive cell line T-47D in which the GI₅₀ was 0.917 μM, while the GI₅₀ of 27 (4-[4-(4′-Chloro-phenylsulfanyl)-phenyl]-thiazol-2-yl ammonium iodide (27)) was 24.4 μM for the T-47D cell line. Compound 27 showed greater selectivity for the estrogen-negative cell line MDA-MB435 with a GI₅₀ of 1.49 μM when compared with greater than 100 μM for 17. The aforementioned selectivity was also seen for compounds 22 and 30 (4-[4′-(p-Toluoxy)phenyl]-thiazol-2-yl ammonium iodide (22) and 4-(4′p -Tolysulfanyl-phenyl)-thiazol-2-yl ammonium iodide (30)) for the same cell lines. These results strongly suggest that estrogen-positive selectivity appears to be achieved using an oxygen linkage whereas compounds with a s linkage are significantly more active against estrogen receptor-negative breast cancer cell types.

Thiazole 20 (4-[4′-(3,4-Dichlorophenoxy)-phenyl]-thiazol-2-yl ammonium iodide (20)) was the only compound to show significant cytotoxicity in the estrogen-negative HS-578T cell line, with a G₅₀ of 2 μM. Low micromolar cytotoxicity was also observed for 28 (4-[4-(3′,4′-Dichloro-phenylsulfanyl)-phenyl]-thiazol-2-yl ammonium iodide (28) which was active in all cell lines except HS 578T with GI₅₀ values in the 1-5 μM range. Compounds 20 and 28 have similar 3,4-dichloro substitutions on their outer phenyl ring and differ only in the heteroatom that links the two rings. Thiazoles 22-24 demonstrated selectivity for the estrogen receptor-positive cell line TA47D with GI₅₀ values of 0.54 μM, 3.91 μM, and 4.90 WA respectively. Compound 30 was selective against the MDA-MB-435 cell line with a GI₅₀ of 1.4 μM. In addition to the selectivity attained with different linkages between the two phenyl rings, these thiazoles also exhibit several distinct structure-activity relationships for substitution on the outer phenyl ring.

For the oxygen linker series, thiazole 22 (4-[4′-p-Toluoxy)phenyl]-thiazol-2-yl ammonium iodide (compound 22)) with an electron-donating para-methyl substituent showed increased efficacy over the electron-withdrawing para-chloro thiazole 17 in all cell lines. An increase in bulk at the para position from methyl to phenyl does not significantly alter activity. Chlorine substitution at the ortho, meta, and para positions was also investigated with compounds 17-19. In most of the cell lines, the best activity was achieved by chloro substitution at the meta position. Substitution at the ortho position showed a slight decrease in activity whereas the para-chloro thiazole had GI₅₀ values greater than 100 μM in all cell lines except T-47D. Although the meta- and para- chloro thiazoles were less active than the ortho-chloro compound, chlorine substitution at both the meta and para positions provided the most active compound out of this series.

While a para electron-donating group was more active (compare GI₅₀ of 22 to that of 17) than a para electron-withdrawing group for the oxygen linker series, the opposite relationship was observed for compounds with the thioether linkage. Generally, for this series of compounds, the para chloro thiazole was more active (compare GI₅₀ of 27 to that of 30) than the para-methyl thiazole.

Thiazoles 17 and 22-24 were selective for the estrogen-positive cell line T-47D. Although these thiazoles may work through a tamoxifen-like mechanism, further studies are in progress for these compounds. However, thiazoles 18, 27, and 30 appear to be active only against estrogen receptor-negative breast cancer thereby ruling out a tamoxifen-like mechanism of action for these structural analogues.

In addition, the thiazoles were evaluated for their ability to disrupt cellular microtubules and for changes in cell cycle distribution as previously described (Tinley et al., Cancer Res., 2003, 63:3211-3220). The results indicate that these compounds are not microtubule or microfilament inhibitors and they do not alter cell cycle distribution.

In summary, a series of 4-aryloxy- and 4-arylsulfanyl-phenyl-2-aminothiazoles have been synthesized with anti-proliferative activity which are useful as anti-cancer agents. Compounds 17, 18, 22, 24, 27, and 30 demonstrated selective cytotoxicity of either estrogen- positive or negative breast cancer cells while compounds 20 and 28 showed low micromolar growth inhibition of most human breast tumor cells. In general, thiazoles with an oxygen linkage showed estrogen receptor-positive selectivity, whereas estrogen receptor-negative selectivity was achieved by thioether linkages. Thiazoles 20 and 28, both with 3,4-dichloro substitutions, exhibited selectivity for the adriamycin-resistant cell line. These thiazoles represent promising lead compounds for the development of selective thiazole-containing breast cancer agents and are candidates for further mechanistic studies.

The disclosures of each and every publication cited herein are hereby incorporated herein by reference in their entirety.

Other methods which were used but not described herein are well known and within the competence of one of ordinary skill in the art of clinical, chemical, cellular, histochemical, biochemical, molecular biology, microbiology and recombinant DNA techniques. For example, other analogues and derivatives of structure I with the ability to inhibit cancer cell proliferation may be prepared by techniques known in the art. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A compound having the general structural formula of structure I 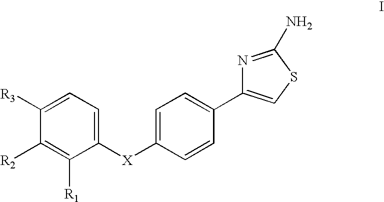
wherein said compound is selected from the group consisting of substituted 4aryloxy and 4-arylsulfanyl-phenyl-2-aminothiazoles comprising structure I, further wherein said compound has anti-cancer activity.

2. The compound of claim 1, wherein:
X is selected from the group consisting of O, S, and NH; and
R₁, R₂, and R₃ are independently selected from the group consisting of H, halo, (C₁-C₄)alkyl, (C₁-C₄)alkoxy, aryl, —O-aryl and (CO)OR₄; and
R₄ is H or (C₁-C₄)alkyl; and
pharmaceutically acceptable salts thereof.

3. The compound of claim 1, wherein:
X is0or S; and
R₁ is H; and
R₂ and R₃ are independently selected from the group consisting of H, Cl, (C₁-C₂)allyl, (C₁-C₂)alkoxy, phenyl, —O-phenyl and (CO)OCH₂CH₃; and
pharmaceutically acceptable salts thereof.

4. The compound of claim 1, wherein:
X is O or S; and
R, is H; and
R₂ and R₃ are independently selected from the group consisting of H, Cl, (C₁-C₂)alkyl, (C₁-C₂)alkoxy, phenyl, —O-phenyl and (CO)OCH₂CH₃; and
pharmaceutically acceptable salts thereof.

5. A compound according to claim 1 selected from the group consisting of 4-(4′-Phenoxyphenyl)-thiazol-2-yl ammonium iodide (16); 4-[4′-(4-Chlorophenoxy)-phenyl]-thiazol-2-yl ammonium iodide (17); 4-[4-(3′-Chlorophenoxy)-phenyl]-thiazol-2-yl ammonium iodide (18); 4-[4-(2′-Chlorophenoxy)-phenyl]-thiazol-2-yl ammonium iodide (19); 4-[4′-(3,4-Dichlorophenoxy)-phenyl]-thiazol-2-yl ammonium iodide (20); 4-[4′-(4-Methoxyphenoxy)-phenyl]-thiazol-2-yl ammonium iodide (21); 4-[4′-(p-Toluoxy)phenyl]-thiazol-2-yl ammonium iodide (22); 4-[4′-(Biphenyl-4-yloxy)-phenyl]-thiazol-2-yl ammonium iodide (23); 4-[4′-(4-Phenoxy-phenoxy)-phenyl]-thiazol-2-yl ammonium iodide (24); 4-[4-(3′-Ethoxycarbonyl-phenoxy)-phenyl]-thiazol-2-yl ammonium iodide (25); 4-(4′-Phenylsulfanyl-phenyl)-thiazol-2-yl ammonium iodide (26); 4-[4-(4′-Chloro-phenylsulfanyl)-phenyl]-thiazol-2-yl ammonium iodide (27); 4-[4-(3′,4′-Dichloro-phenylsulfanyl)-phenyl]-thiazol-2-yl ammonium iodide (28); 4-[4-(4′-Methoxy-phenylsulfanyl)-phenyl]-thiazol-2-yl ammonium iodide (29); and 4-(4′-p -Tolylsulfanyl-phenyl)-thiazol-2-yl ammonium iodide (30).

6. A pharmaceutical composition comprising at least one compound according to claims 1, 2, 3, 4, or 5, and a pharmaceutically acceptable carrier.

7. A method of preparing substituted 4-aryloxy and 4-arylsulfanyl-phenyl-2-aminothiazole compounds with anti-cancer activity comprising the structural formula of structure I: 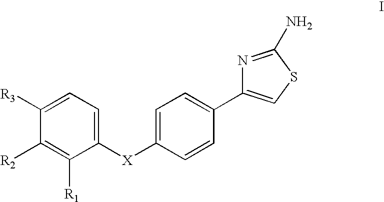
said method comprising preparing said compound according to scheme 1:

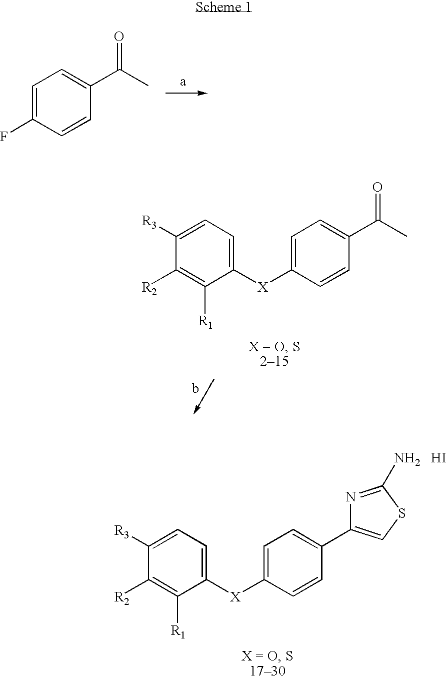

8. A compound prepared by the method of claim 7, wherein:
X is selected from the group consisting of O, S, and NH; and
R₁, R₂, and R₃ are independently selected from the group consisting of X halo, (C₁-C₄)alkyl, (C₁-C₄)alkoxy, aryl, —O-aryl and (CO)OR₄; and
R₄ is H or (C₁-C₄)alkyl; and
pharmaceutically acceptable salts thereof.

9. A compound prepared by the method of claim 7, wherein:
X is O or S; and
R₁ is H; and
R₂ and R₃ are independently selected from the group consisting of H, Cl, (C₁-C₂)alkyl, (C₁-C₂)alkoxy, phenyl, —O-phenyl and (CO)OCH₂CH₃; and
pharmaceutically acceptable salts thereof.

10. A compound prepared by the method of claim 7, wherein:
X is O or S; and
R₁ is H; and
R₂ and R₃ are independently selected from the group consisting of H, Cl, (C₁-C₂)alkyl, (C₁-C₂)alkoxy, phenyl, —O-phenyl and (CO)OCH₂CH₃; and
pharmaceutically acceptable salts thereof.

11. The method of claim 7, wherein a compound prepared by said method is selected from the group consisting of:
4-(4′-Phenoxyphenyl)-thiazol-2-yl ammonium iodide (16); 4-[4′-(4-Chlorophenoxy)-phenyl]-thiazol-2-yl ammonium iodide (17); 4-[4-(3′-Chlorophenoxy)-phenyl]-thiazol-2-yl ammonium iodide (18); 4-[4-(2′-Chlorophenoxy)-phenyl]-thiazol-2-yl ammonium iodide (19); 4-[4′-(3,4-Dichlorophenoxy)-phenyl]-thiazol-2-yl ammonium iodide (20); 4-[4′-(4-Methoxyphenoxy)-phenyl]-thiazol-2-yl ammonium iodide (21); 4-[4′-(p-Toluoxy)phenyl]-thiazol-2-yl ammonium iodide (22); 4-[4′-(Biphenyl-4-yloxy)-phenyl]-thiazol-2-yl ammonium iodide (23); 4-[4′-(4-Phenoxy-phenoxy)-phenyl]-thiazol-2-yl ammonium iodide (24); 4-[4-(3′-Ethoxycarbonyl-phenoxy)-phenyl]-thiazol-2-yl ammonium iodide (25); 4-(4′-Phenylsulfanyl-phenyl)-thiazol-2-yl ammonium iodide (26); 4-[4-(4′-Chloro-phenylsulfanyl)-phenyl]-thiazol-2-yl ammonium iodide (27); 4-[4-(3′,4′-Dichloro-phenylsulfanyl)-phenyl]-thiazol-2-yl ammonium iodide (28); 4-[4-(4′-Methoxy-phenylsulfanyl)-phenyl]-thiazol-2-yl ammonium iodide (29); and 4-(4′p-Tolylsulfanyl-phenyl)-thiazol-2-yl ammonium iodide (30).

12. A method of treating cancer in a subject in need thereof, said method comprising administering to said subject an effective amount of a pharmaceutical composition comprising at least one compound according to claims 1, 2, 3, 4, or 5, or pharmaceutically acceptable salts thereof.

13. The method of claim 12, wherein said cancer is selected from the group consisting of breast cancer, leukemia, non-small cell lung cancer, colon cancer, cancer of the central nervous system, melanoma, ovarian cancer, renal cancer, and prostate cancer.

14. The method of claim 12, wherein said breast cancer is selected from the group consisting of estrogen receptor positive, estrogen receptor negative, and adriamycin-resistant breast cancer.

15. The method of claim 12, wherein said composition is administered intravenously.

16. The method of claim 12, wherein said composition is administered intra-tumorally.

17. The method of claim 12, wherein said subject is a human.

18. A method of inhibiting proliferation of cancer cells, said method comprising contacting said cells with a composition comprising at least one compound of claims 1, 2, 3, 4, or 5.

19. The method of claim 18, wherein said cancer cell is selected from the group consisting of breast cancer, leukemia, non-small cell lung cancer, colon cancer, cancer of the central nervous system, melanoma, ovarian cancer, renal cancer, and prostate cancer cells.

20. The method of claim 18, wherein said breast cancer cell is selected from the group consisting of estrogen receptor positive, estrogen receptor negative, and adriamycin-resistant breast cancer cells.

21. A kit for administering a compound which has anti-cancer activity to a subject in need of such treatment, said kit comprising a pharmaceutical composition comprising at least one compound of claims 1, 2, 3, 4, or 5, an applicator, and an instructional material for the use thereof.
